(19) United States
(12) Patent Application Publication
(10) Pub. No.: US 2014/0072799 A1
(43) Pub. Date: Mar. 13, 2014

(54) ARTICLE AND A METHOD OF MAKING AN ARTICLE

(71) Applicant: Thomas Campbell Prentice, Abingdon (GB)

(72) Inventor: Thomas Campbell Prentice, Abingdon (GB)

(73) Assignee: ZIRCOTEC LIMITED, Abingdon, Oxfordshire (GB)

(21) Appl. No.: 14/005,191

(22) PCT Filed: Mar. 14, 2012
(86) PCT No.: PCT/GB12/50563

§ 371 Date: Nov. 20, 2013

(30) Foreign Application Priority Data
Mar. 14, 2011 (GB) 1104256.1

(51) Int. Cl. C23C 4/10; B60B 21/08; A61L 27/30

(52) U.S. Cl. CPC C23C 4/105; A61L 27/306; B60B 21/08
USPC 428/339; 427/453; 427/446; 428/702; 428/472; 428/472.2; 301/6.9

(57) ABSTRACT

An article, such as a bicycle wheel (10), includes a substrate, at least a surface of the substrate being made of or containing a composite material comprising fibres dispersed in an organic matrix, for example a carbon fibre composite. A thermal sprayed first layer of coating material is provided on the surface, such as the braking area of the rim of the bicycle, the coating material of the first layer wholly comprising inorganic material and including at least one of the group comprising TiO₂, titanate, Al₂O₃ and aluminate.

The invention relates to an article and a method of making an article.

It is known to coat metal parts, for example engine parts, by plasma spraying with a layer of ceramic or metal material. In plasma spraying, the material to be deposited is melted and propelled towards the substrate to be coated. The plasma jet temperature may be of the order of 20,000 K.

According to a first aspect of the present invention there is provided an article, the article including a substrate, at least a surface of the substrate being made of or containing an organic material or a composite material comprising fibres dispersed in an organic matrix, and a thermal sprayed first layer of coating material on the surface, the coating material of the first layer wholly comprising inorganic material and including at least one of the group comprising TiO₂, titanate, Al₂O₃, and aluminate.

It has been found that, surprisingly, TiO₂, titanate, Al₂O₃, and aluminate can be thermally sprayed directly onto an organic or fibre composite material with an organic matrix, without detrimental damage to the material, and that a well adhered coating results.

According to a second aspect of the present invention, there is provided an article, the article including a substrate, at least a surface of the substrate being made of or containing an organic material, a thermal sprayed first layer of coating material on the surface, the coating material of the first layer wholly comprising inorganic material and including at least one of the group comprising TiO₂, titanate, Al₂O₃, and aluminate, the article further comprising a further layer on the first layer.

It has been found that, surprisingly, TiO₂, titanate, Al₂O₃, and aluminate not only can be thermally sprayed directly onto a composite material with an organic matrix, without detrimental damage to the material, but also that the resulting coating forms a very effective bond coat for a further layer.

According to a third aspect of the present invention, there is provided an article, the article including a substrate, at least a surface of the substrate being made of or containing an organic material, and a thermal sprayed first layer of coating material on the surface, and a further layer on the first layer, the coating material of the first layer wholly comprising inorganic material and including at least one of the group comprising TiO₂, titanate, Al₂O₃, and aluminate, the further layer comprising at least 50 wt-% of at least one of zirconia, titania and alumina.

According to a fourth aspect of the present invention, there is provided a bicycle wheel, the bicycle wheel including a rim for a bicycle tyre, the rim including an outer surface wholly or principally of carbon fibre composite material, the wheel including on the outer surface of the rim a thermal sprayed first layer of coating material, the coating material of the first layer wholly comprising inorganic material and including at least one of the group comprising TiO₂, titanate, Al₂O₃, and aluminate.

According to a fifth aspect of the present invention, there is provided a golf club, the head of the golf club being wholly or principally of carbon fibre composite material, at least one of the striking face and the ground engaging face of the golf club including thereon a first layer of coating material, the coating material of the first layer wholly comprising inorganic material and including at least one of the group comprising TiO₂, titanate, Al₂O₃, and aluminate.

According to a sixth aspect of the present invention, there is provided a bone or tooth implant comprising a substrate, the substrate being wholly or principally made of carbon fibre composite material, a surface of the substrate which is arranged to engage bone or tooth when implanted having a thermal sprayed first layer of coating material, the coating material of the first layer wholly comprising inorganic material and including at least one of the group comprising TiO₂, titanate, Al₂O₃, and aluminate.

According to a seventh aspect of the present invention, there is provided a junction box for electromagnetic shielding, the box including a surface wholly or principally of organic or carbon fibre composite material, the box including on the outer surface of the rim a thermal sprayed first layer of coating material, the coating material of the first layer wholly comprising inorganic material and including at least one of the group comprising TiO₂, titanate, Al₂O₃, and aluminate.

According to an eighth aspect of the present invention, there is provided a carbon fibre composite mould for moulding carbon fibre composite material, the mould comprising an inner surface wholly or principally of carbon fibre composite material, the mould including on its inner surface a thermal sprayed first layer of coating material, the coating material of the first layer wholly comprising inorganic material and including at least one of the group comprising TiO₂, titanate, Al₂O₃, and aluminate.

According to an ninth aspect of the present invention, there is provided a method of coating a substrate surface made of or containing organic material or a composite material comprising fibres dispersed in an organic matrix, the method comprising thermal spraying the surface with a first layer of coating material, the coating material of the first layer wholly comprising inorganic material and including at least one of the group comprising TiO₂, titanate, Al₂O₃, and aluminate.

According to a tenth aspect of the present invention, there is provided a method of coating a substrate surface made of or containing an organic material, the method comprising thermal spraying the surface with a first layer of coating material, the coating material of the first layer wholly comprising inorganic material and including at least one of the group comprising TiO₂, titanate, Al₂O₃, and aluminate, the method further comprising the step of depositing a further layer on the first layer.

According to an eleventh aspect of the present invention, there is provided a method of coating a substrate surface made of or containing an organic material, the method comprising thermal spraying the surface with a first layer of coating material, the coating material of the first layer wholly comprising inorganic material and including at least one of the group comprising TiO₂, titanate, Al₂O₃, and aluminate, and depositing a further layer on the first layer, the further layer comprising at least 50 wt-% of zirconia, titania, and/or alumina.

According to an twelfth aspect of the present invention, there is provided a method of making a bicycle wheel, the bicycle wheel including a rim for a bicycle tyre, the rim including an outer surface wholly or principally of carbon fibre composite material, the method comprising depositing by thermal spraying on the outer surface of the rim a first layer of coating material, the coating material of the first layer wholly comprising inorganic material and including at least one of the group comprising TiO₂, titanate, Al₂O₃, and aluminate.

According to an thirteenth aspect of the present invention, there is provided a method of making a golf club, the head of the golf club being wholly or principally of carbon fibre composite material, the method comprising depositing on at least one of the striking face and the ground engaging face of the golf club a first layer of coating material, the coating material of the first layer wholly comprising inorganic material and including at least one of the group comprising TiO₂, titanate, Al₂O₃, and aluminate.

According to an fourteenth aspect of the present invention, there is provided a method of making a bone or tooth implant comprising a substrate, the substrate being wholly or principally made of carbon fibre composite material, the method comprising thermal spraying a surface of the substrate which is arranged to engage bone or tooth when implanted with a first layer of coating material, the coating material of the first layer wholly comprising inorganic material and including at least one of the group comprising TiO₂, titanate, Al₂O₃, and aluminate, and thermal spraying a top layer of hydroxyapatite.

According to an fifteenth aspect of the present invention, there is provided a method of making a junction box for electromagnetic shielding, the box including a surface wholly or principally of carbon fibre composite material, the method including depositing by thermal spraying a first layer of coating material on the surface, the coating material of the first layer wholly comprising inorganic material and including at least one of the group comprising TiO₂, titanate, Al₂O₃, and aluminate.

According to an sixteenth aspect of the present invention, there is provided a method of making a carbon fibre composite mould for moulding carbon fibre composite material, the mould comprising an inner surface, the method including depositing by thermal spraying a first layer of coating material on the surface, the coating material of the first layer wholly comprising inorganic material and including at least one of the group comprising TiO₂, titanate, Al₂O₃, and aluminate.

Embodiments of the invention will now be described by way of example and with reference to the accompanying drawings, in which:

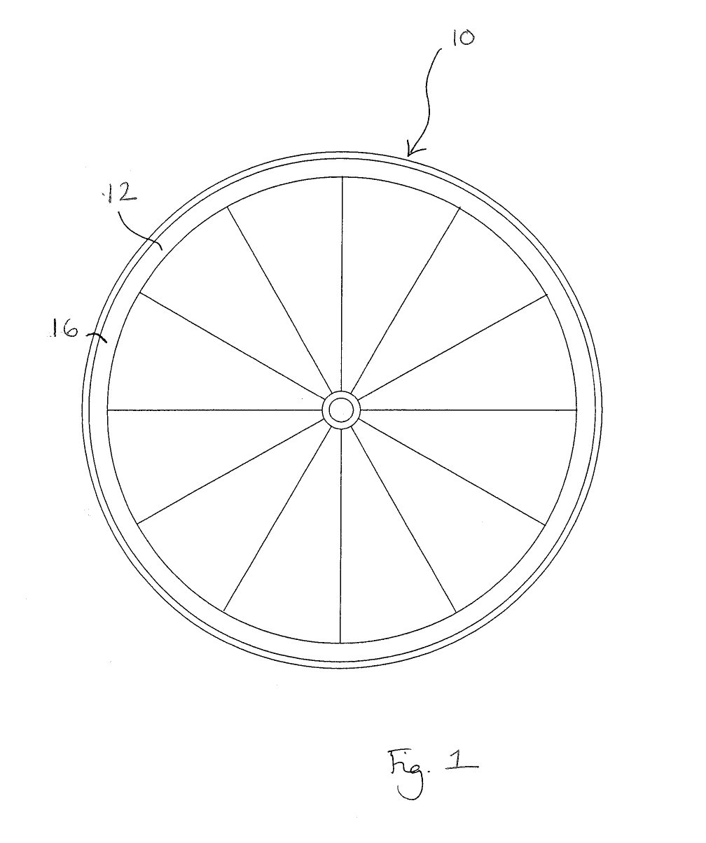

FIG. 1 shows a bicycle wheel according to Embodiments 1 and 2 of the present invention;

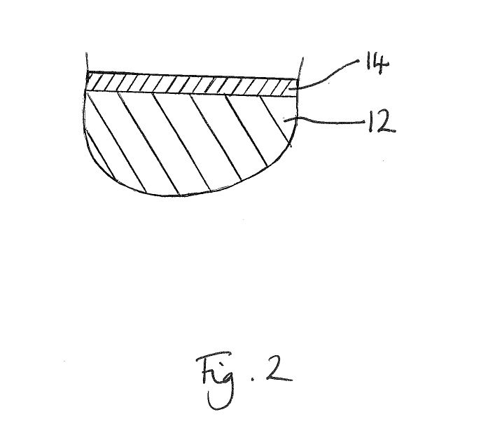

FIG. 2 is a schematic view in cross section of the rim of Embodiment 1 with a coating layer;

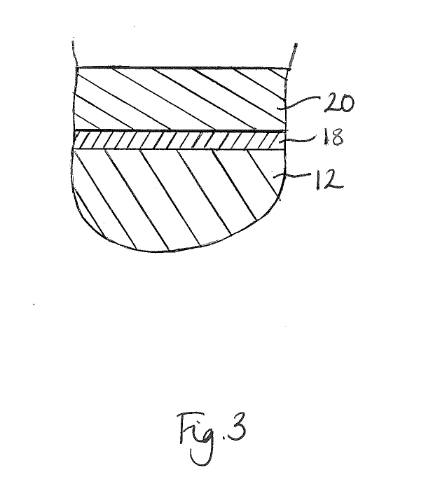

FIG. 3 is a schematic view in cross section of the rim of Embodiment 1 with a coating layer;

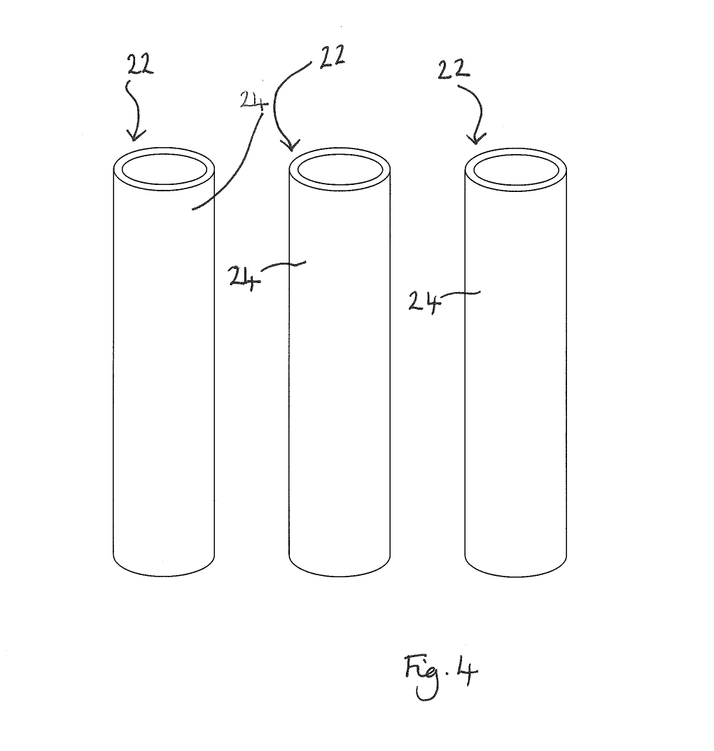

FIG. 4 shows a glass fibre epoxy tubes according to Embodiment 3 of the present invention;

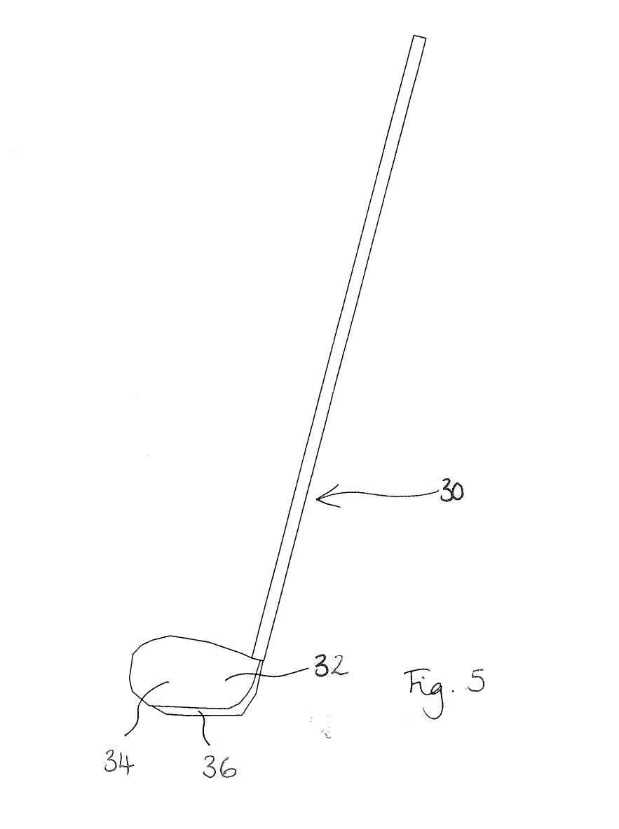

FIG. 5 shows a golf club according to Embodiment 4 of the present invention;

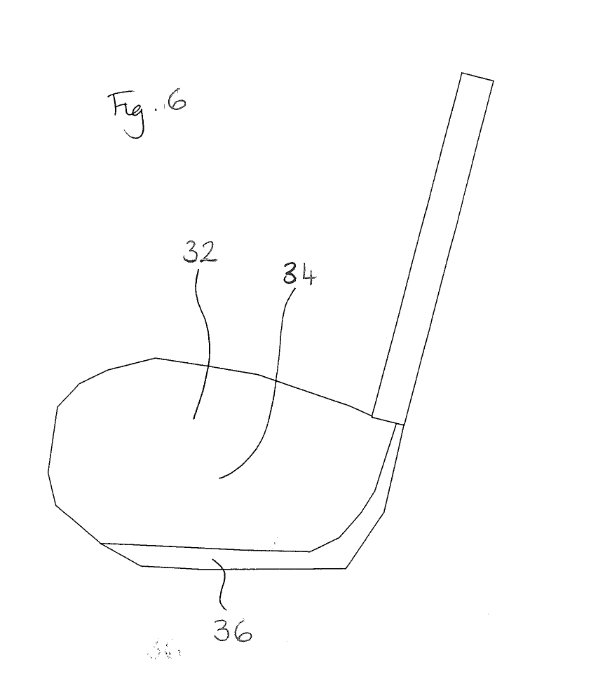

FIG. 6 shows the head of the golf club of FIG. 5, in fragmentary detail;

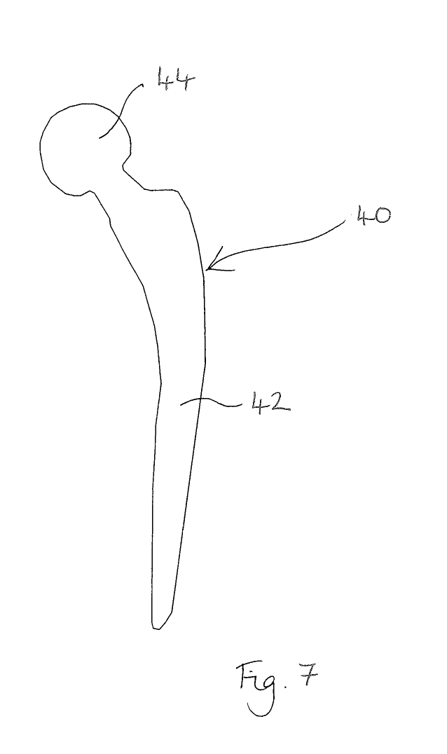

FIG. 7 shows a hip prosthesis according to Embodiment 5 of the present invention;

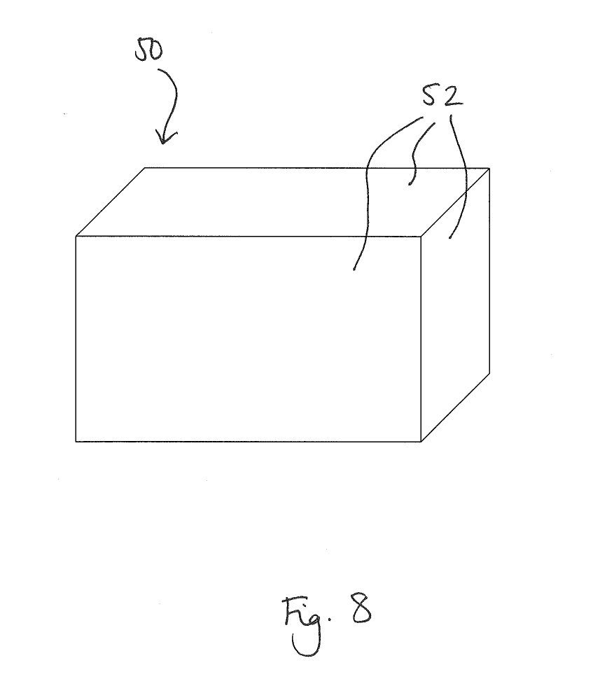

FIG. 8 shows a junction box according to Embodiment 6 or the present invention; and

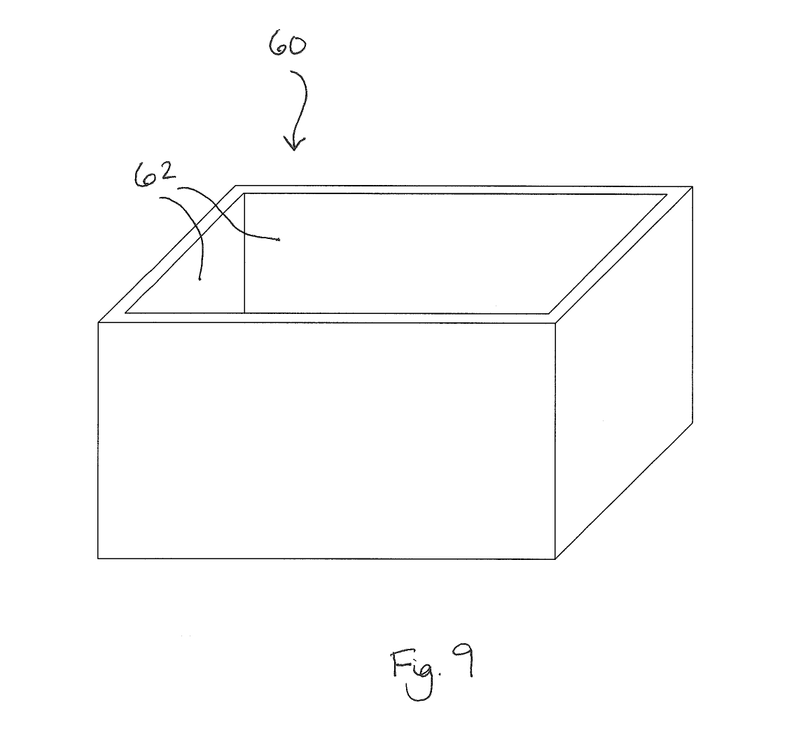

FIG. 9 shows a mould according to Embodiment 7 of the present invention.

### EMBODIMENT 1

A carbon fibre reinforced plastic bicycle wheel 10, as shown in FIG. 1, quickly suffered wear when brakes pads were applied to the rim 12.

A coating layer 14 comprising 100 wt-% titania (TiO₂) was plasma sprayed onto the braking area 16, namely the area of the rim to be contacted by a brake block in use.

The coating layer was deposited to a thickness of ˜50 μm. The plasma spray parameters used were nitrogen 60 slpm, hydrogen 5 slpm, current 400 Amps, carrier gas 5 slpm, spray distance 100 mm, powder flow 45 g/min, surface speed ˜5 ms⁻¹. A schematic view of the rim and coating in cross section is shown in FIG. 2.

The wear resistance of the wheel rim 12 was increased by the coating 14. The adherence of the coating layer 14 to the braking area 14 was better than metal coating layers.

### EMBODIMENT 2

A carbon fibre reinforced plastic bicycle wheel 10 was used, the same as that shown in FIG. 1.

A bond coat 18 comprising 100 wt-% titania (TiO₂) was plasma sprayed onto the braking area 16. The bond coat 18 was deposited to a thickness of ˜50 μm. The plasma spray parameters used were nitrogen 60 slpm, hydrogen 5 slpm, current 400 Amps, carrier gas 5 slpm, spray distance 100 mm, powder flow 45 g/min, surface speed ˜5 ms⁻¹ .

An Al₂O₃/TiO₂ ceramic layer 20 was then applied on top of the bond coat 18 by plasma spraying. The ceramic layer 20 was applied to a thickness of ˜150 μm. The plasma spray parameters used were Nitrogen 60 slpm, hydrogen 5 slpm, current 500 Amps, carrier gas 5 slpm, spray distance 75 mm, powder flow 45 g/min, ceramic powder particle size 50 to 90 micrometres.

A schematic view of the rim and coatings in cross section is shown in FIG. 3.

The wear resistance of the wheel rim 12 was increased by the coatings. The adherence of the bond coat to the braking area 14 was increased compared with metal bond coats.

### EMBODIMENT 3

Glass fibre epoxy tubes 22, as shown in FIG. 4, were plasma sprayed on their outer surfaces 24, with a bond coat of Al₂O₃/TiO₂ to a thickness of 50 μm. The plasma spray parameters used were Nitrogen 75 lpm, hydrogen 5 lpm, current 500 Amps, spray distance 75 mm, surface speed ˜2 ms⁻¹

An MgZnO₃ ceramic layer was then applied on top of the bond coat by plasma spraying, to a thickness of 200 μm. The plasma spray parameters used were Nitrogen 75 lpm, hydrogen 5 lpm, current 500 Amps, spray distance 75 mm, surface speed ˜2 ms⁻¹

### EMBODIMENT 4

A golf club 30, as shown in FIGS. 5 and 6, with a carbon fibre reinforced plastic golf club head 32 was coated with titania ceramic on the striking face 34 of the head 32 and the bottom 36 of the head 32. The spray system was set to work in nitrogen and titania powder feed set to spray at 30 gm/min. Nitrogen flow was preset to 50 litres/min and current to 300 Amps. The robot was programmed to operate a ladder type spray pattern, at a stand off distance of 100 mm from the surface being coated the surface was rotated. In this way, titania bond coat of approximately 25 μm thickness was applied. A second coat was then applied in the same way to provide a layer with a total coating thickness of 50 μm. To avoid impact damage, a 50 μm molybdenum layer was applied to the titania layer, using a Metco™ 9 MB plasma spray gun, mounted on a Staubli™ robot, using the following parameters:

Powder feed rate—25 g/min

Nitrogen flow—80 scfh

Hydrogen flow—10 scfh

Current—500 A

Spray distance—100 mm

Traverse rate—150 mm/s

The surface integrity of the ceramic was enhanced by the addition of this thin molybdenum coating, so that if any damage was done to the ceramic, the metal layer would hold it together, preventing spalling.

### EMBODIMENT 5

A carbon fibre reinforced plastic hip prosthesis 40, comprising a stem 42 and a ball 44, as shown in FIG. 7, was plasma sprayed with a 150 μm titania layer. A 150 μm hydroxyapatite layer was then plasma sprayed onto the on the surface of the stem 42. The hydroxyapatite coating helped to promote bone ingrowth and enhance the fixation of the implant to the femur.

### EMBODIMENT 6

A carbon fibre resin composite junction box 50, as shown in FIG. 8, for electromagnetic shielding in aircraft was coated on its outer surface 52 with a coating layer of Al₂O₃/TiO₂ by plasma spraying. The coating layer 52 provided a tough protective layer for the box without the need for a metal layer.

### EMBODIMENT 7

A lightweight mould 60, as shown schematically in FIG. 9, for moulding carbon fibre resin composite structures, for example for large aircraft parts, for example 5 m long or more, is made of carbon fibre resin composite. The inner surface 62 of the mould can easily be damaged on removal of the part being moulded.

In this embodiment, the inner surface 62 of the mould 60 was plasma sprayed with a coating layer of Al₂O₃/TiO₂. The ceramic layer provides a tough, wear resistant inner surface for the mould, and prolongs its useful lifespan.

Titania/alumina can be used as a bond coat for an electrically conductive metallic outer layer, the outer layer providing electromagnetic compatibility shielding (EMCS) with the bond coat providing electrical insulation between the outer layer and the substrate thereby avoiding electrolytic corrosion, spalling and other issues associated with any contact between the metal and substrate.

Titania can be used as a compatible bond coat for the addition of a titania based hard-ceramic outer layer, the latter being used to provide wear resistance. This is applicable to components such as print rollers, in particular lightweight print rollers.

The use of a ceramic (for example, titania/alumina) bond coat, instead of a metal bond, means that coatings can be used on a non-conducting substrate such as carbon composite and plastic in an RF environment.

A bond coat such as a titania/alumina ceramic coating can be used on carbon composite and plastics components where parts are required to be transparent to electromagnetic radiation, eg. mobile communication devices where a hard wearing surface is required on the outside of a structural plastic or composite casing.

Titania and titania/alumina have been proven to be applicable as bond coats on various non-loaded plastics such as PEEK and polyimide.

1. An article, at least a surface of the article being made of an organic material or a composite material comprising fibres dispersed in an organic matrix, and a thermal sprayed first layer of coating material on the surface, the coating material of the first layer wholly comprising inorganic material and being wholly or principally one of the group comprising TiO₂ and titanate.

2. (canceled)

3. An article according to claim 1, wherein the organic material or matrix is made of plastics material.

4. An article according to claim 1, further comprising a further thermally_sprayed layer on the first layer.

5-7. (canceled)

8. An article according to claim 4, wherein the further layer is wholly or principally made of ceramic material.

9. An article according to claim 4, wherein the further layer is a blend including one of the group comprising TiO₂, titanate, Al₂O₃ and aluminate.

10-27. (canceled)

28. An article according to claim 1, wherein the first layer is up to 300 micrometres in thickness.

29. (canceled)

30. An article according to claim 1, wherein the first layer is greater than 75 micrometres in thickness,

31-32. (canceled)

33. An article according to claim 1, wherein the article is a bicycle wheel and the first layer is on the braking area of the rim of the wheel.

34. (canceled)

35. An article according to claim 1, wherein the article is a medical implant.

36. An article according to claim 35, wherein the article is a bone or ooth implant.

37. An article according to claim 35, wherein there is a further layer on the first layer, and the further layer is wholly or principally of titanium.

38. An article according to claim 37, wherein there is an additional layer as a top layer which is wholly or principally of hydroxyapatite.

39-50. (canceled)

51. A method of coating a substrate surface made of or containing organic material or a composite material comprising fibres dispersed in an organic matrix, the method comprising thermal spraying the surface with a first layer of coating material, the coating material of the first layer wholly comprising inorganic material and being wholly or principally one of the group comprising TiO₂ and titanate.

52-80. (canceled)

81. A method according to claim 51, wherein the method includes the step of cooling the substrate whilst the first layer is being deposited.

82. (canceled)

83. A method according to claim 81, wherein the step of cooling the substrate comprises passing an air flow up to 1 m³s⁻¹ over the substrate.

84-115. (canceled)

116. An article as claimed in claim 1, wherein the organic material or matrix is PEEK.

117. An article as claimed in claim 3, wherein the first, layer provides electrical insulation between the article and the further layer.

118. An article, at least a surface of the article being made of an organic material or a composite material comprising fibres dispersed in an organic matrix, and a thermal sprayed first layer of coating material on the surface, the coating material of the first layer wholly comprising TiO₂.
